(19) United States
(12) Patent Application Publication
(10) Pub. No.: US 2015/0209268 A1
(43) Pub. Date: Jul. 30, 2015

(54) Use of Starfruit Extract as a CPT-1 Modulator and Compositions Thereof

(71) Applicant: AVON PRODUCTS, INC., Suffern, NY (US)

(72) Inventors: Cheng S. Hwang, New Milford, NY (US); Sungham Yim, Hackensack, NJ (US); Uma Santhanam, Tenafly, NJ (US); John W. Lyga, Basking Ridge, NJ (US); M. Jean Paufique, Saint Viance (FR)

(21) Appl. No.: 14/372,651

(22) PCT Filed: Feb. 29, 2012
(86) PCT No.: PCT/US12/27065

§ 371 Date: Jul. 16, 2014

Related U.S. Application Data
(60) Provisional application No. 61/604,870, filed on Feb. 29, 2012.

(51) Int. Cl. A61K 8/97; A61Q 19/00; A61Q 19/06

(52) U.S. Cl. CPC A61K 8/97; A61Q 19/06; A61Q 19/008; A61K 2800/78

(57) ABSTRACT

The present invention describes methods for improving the appearance of skin, particularly, treating, ameliorating, preventing, delaying, and/or improving one or more signs of excess accumulation and/or production of subcutaneous fat, such as cellulite, and conditions related thereto, by topically applying compositions comprising Carnitine Palmitoyl Transferase-1 (CPT-1) stimulating aqueous extract of the leaf of Averrhoa carambola, optionally with other anti-lipid agents.

### FIELD OF THE INVENTION

The present invention relates generally to compositions containing Averrhoa Carambola (also known as starfruit) leaf extract for topical application to the skin that modulate the expression of Carnitine Palmitoyl Transferase-1 (CPT-1). The compositions of the present invention comprise at least one anti-lipid agent and provide benefits to the skin, in particular, by improving the condition and appearance of skin affected by cellulite. More particularly, the compositions of the present invention comprise an aqueous starfruit extract that stimulates the expression of CPT-1, an important enzyme for the oxidation of fat.

### BACKGROUND OF THE INVENTION

Consumers continually seek to improve the appearance of their skin, and in particular seek to improve the appearance of skin affected by unwanted deposition and/or accumulation of fat, including cellulite. There is active interest in the cosmetics industry to develop products that may be applied topically to the skin to provide anti-cellulite and anti-adiposity benefits, as well as other anti-lipid benefits. Cosmetic products that enhance the appearance of skin are increasingly in demand as consumers increasingly seek to mitigate and delay signs of excess accumulation and/or production of subcutaneous fat.

Cellulite is the lumpy, uneven type of subcutaneous fat that tends to accumulate on the buttocks, thighs, and limbs of many women. It is considered unsightly because it gives the tissues underlying the skin an “orange peel” or “cottage cheese” look. Compressing the skin, as when sitting or crossing the legs, produces a “mattress appearance” with bulging and pitting of the fatty layer. Nodules of fat may be felt trapped within hardened connective tissue. The histology of cellulite-affected skin indicates that cellulite results from a combination of enlarged fat tissue and weak dermal structure and connective tissue septa. Excess fat accumulation increases the volume of adipocytes, which bulge into a weakened dermis to create the characteristic irregularities in the appearance of the epidermal surface. A number of factors can cause cellulite including, e.g., hereditary, intestinal, circulatory, lymphatic, hormonal, and lifestyle factors. Dieting to decrease fat intake, exercise to increase fat metabolism and prevent the build up of cellulite, and massage and hydrotherapy to stimulate lymphatic drainage can help reduce the appearance of cellulite. Nonetheless, these means for combating cellulite or subcutaneous fat are limited, and the need remains for additional approaches.

There is a need in the art for compositions and methods for improving the appearance of skin, including the treatment, control, management, amelioration, prevention, inhibition, delay, and/or reduction of excess accumulation and/or production of subcutaneous fat, including cellulite, acne, and/or oily skin.

It is therefore an object of the invention to provide new ingredients to treat, ameliorate, prevent, inhibit, delay, and/or reduce the signs of excess accumulation and/or production of subcutaneous fat. Novel methods and compositions, as well as their mode(s) of action, are disclosed herein for treating conditions related to excess accumulation and/or production of subcutaneous fat, including cellulite, as well as skin formulations comprising same, and other personal care products for the skin.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE INVENTION

The protrusion of enlarged fat tissue into the dermis is one of the major factors contributing to the appearance of cellulite. One of the approaches to improve cellulite is to stimulate fat breakdown and reduce the amount of fat and/or lipids in the adipocytes, or fat cells. Carnitine Palmitoyl Transferase-1 (CPT-1) is a mitochondrial enzyme that catalyzes the conjugation of carnitine to fatty acids, which is the rate-limiting step of fatty acid oxidation (fatty acid breakdown). Without wishing to be bound by theory, it is believed that an increase in CPT-1 expression leads to a reduction in lipid accumulation in fat cells, which in turn decreases the size of adipose tissue and helps improve the appearance of skin affected by cellulite.

Prior to this invention, it was not known that a stimulator of CPT-1 expression could reduce lipid accumulation in fat cells. Surprisingly, it was discovered that aqueous extracts of the leaf of Averrhoa carambola (also known as “starfruit,” hereinafter used interchangeably) stimulate CPT-1 expression and lead to reduced lipid accumulation in fat cells. In particular, compositions comprising aqueous extracts of the leaf of Averrhoa carambola have surprisingly been found to reduce fat accumulation and adipocyte differentiation, offering combined mechanisms of action for combating unwanted subcutaneous fat, and cellulite in particular. Furthermore, starfruit extract can be beneficial in treating other skin conditions characterized by excess lipids, e.g., acne or oily skin.

Averrhoa carambola, also known as Carambola or starfruit, is a species of woody plant in the family Oxalidaceae. This evergreen tree is native to Southeast Asia and the Indian subcontinent. Averrhoa carambola is a small tree or shrub, with rose to red-purple flowers. The flowers are small and bell-shaped, with five petals that have whitish edges. The flowers are often produced year round under tropical conditions. The tree is cultivated in tropical and semitropical regions for its edible fruits and for medicinal uses.

While Averrhoa carambola leaf extract has been described in the treatment of wrinkles (JP 2003/3009893, JP 2002/226323, and JP 2003/055244), and also in the inhibition of testosterone 5-a-reductase (JP 2002/241296A), Averrhoa carambola extract has never been described for combating unwanted subcutaneous fat and/or cellulite.

In one aspect of the invention, cosmetic compositions (or personal care products) are provided for improving the appearance of skin comprising a Carnitine Palmitoyl Transferase-1 (CPT-1) stimulator in a cosmetically acceptable vehicle in an amount effective to decrease adipocyte differentiation and/or intracellular triglyceride production and/or accumulation in adipocytes in said area of skin. In some embodiments, the CPT-1 stimulator is an Averrhoa carambola extract, in particular, an aqueous extract of the leaves, stems, and flowers (or combinations thereof) of the plant. In one aspect of the invention, the CPT-1 stimulator is an aqueous extract of leaves of Averrhoa carambola and stimulates CPT-1 gene expression.

In some embodiments of the invention, methods for treating a skin condition characterized by excess lipids, oily skin and/or oily scalp are provided, comprising topically applying to skin in need thereof an effective amount of a CPT-1 stimulating aqueous extract of the leaf of Averrhoa carambola in a cosmetically acceptable vehicle for a time sufficient to improve the appearance of said skin.

In some embodiments, the cosmetic composition further comprises at least one other anti-lipid agent, the anti-lipid agent being selected from the group consisting of a phophodiesterase inhibitor, an adenylate cyclase activator, a lipolysis stimulator, a beta-adrenergic receptor agonist, an alpha-2-adrenergic receptor antagonist, a xanthine analog, forskolin, a forskohlii extract, a hawthorne extract, a cola extract, isoproterenol, yohimbine, Ginkgo biloba extract, perilla oil, caffeine, a collagen stimulator, an elastin stimulator, carnitine, creatine, and combinations thereof.

In another aspect, the invention relates to methods for improving the appearance of skin affected by unwanted subcutaneous fat, such as cellulite, comprising topically applying to the skin a cosmetic composition comprising one or more CPT-1 modulators in a cosmetically acceptable vehicle in an amount effective to decrease adipocyte differentiation and/or intracellular triglyceride production and/or accumulation in adipocytes in said area of skin. In some embodiments, the CPT-1 stimulator is an Averrhoa carambola extract, in particular, an aqueous and/or ethanol extract of the leaves, stems and flowers of the plant, particularly the leaves. For example, an effective amount of the extract of Averrhoa carambola in a cosmetically acceptable vehicle can be applied to skin for a length of time sufficient to improve the appearance of the skin.

In some embodiments, the composition is applied to cellulite found on at least one of the thigh, buttocks, abdomen, hip, or upper arm region. In some embodiments, the composition is left on the area of application, e.g., as a leave-on composition. In some embodiments, the composition is applied according to a treatment regimen, such as at least once a day application for a period of at least 4 weeks. In some embodiments the treatment regimen comprises at least once a day application for a period of at least 2 weeks. In particular embodiments, methods are provided wherein the time sufficient to improve the appearance of said skin comprises a period of at least 2 weeks, and wherein said aqueous extract is applied at least once a day. In other embodiments, the composition is applied two or three times a day.

Also provided is a method for reducing the re-occurrence of cellulite in an area previously affected by cellulite, comprising topically applying thereto an effective amount of a CPT-1 stimulating aqueous extract of the leaf of Averrhoa carambola in a cosmetically acceptable vehicle.

Yet another aspect relates to methods for reducing unwanted fat deposition comprising topically applying to an area affected by unwanted fat deposition and/or accumulation an effective amount of a CPT-1 stimulating aqueous extract of the leaf of Averrhoa carambola in a cosmetically acceptable vehicle, for a time sufficient to reduce the unwanted fat.

Another embodiment of the invention provides a personal care or cosmetic composition for treating a skin condition characterized by excess lipids comprising an effective amount of a CPT-1 stimulating aqueous extract of the leaf of Averrhoa carambola in a cosmetically acceptable vehicle.

Another embodiment of the invention provides compositions for treating a skin condition characterized by excess lipids, comprising an effective amount of a CPT-1 stimulating aqueous extract of the leaf of Averrhoa carambola in a cosmetically acceptable vehicle, wherein said aqueous extract has an HPLC analysis according to FIG. 1.

Another embodiment of the invention provides compositions for treating a skin condition characterized by excess lipids, comprising an effective amount of a CPT-1 stimulating aqueous extract of the leaf of Averrhoa carambola in a cosmetically acceptable vehicle, wherein said aqueous extract comprises less than 0.1% by weight phenolic compounds and at least 20% by weight carbohydrates.

These and other aspects of the present invention will be better understood by reference to the following detailed description.

### BRIEF DESCRIPTION OF THE DRAWINGS

FIG. 1 shows an HPLC analysis of an Averrhoa carambola leaf extract that stimulates CPT-1 according to the invention.

FIG. 2 shows an HPLC analysis of an Averrhoa carambola leaf extract that down-regulates CPT-1.

### DETAILED DESCRIPTION

It has surprisingly been found that compositions comprising one or more substances that stimulate Carnitine Palmitoyl Transferase-1 (CPT-1) expression markedly improve the appearance of skin affected by unwanted fat deposition and/or accumulation, including skin affected by cellulite, when topically applied thereto. In particular, cosmetic compositions comprising extracts, preferably aqueous extracts, of Averrhoa carambola can be used in such methods to improve the appearance of skin affected by cellulite, as well as to reduce the re-occurrence of cellulite in previously-affected area, and/or to reduce obesity in areas affected by unwanted fat accumulation and/or deposition.

CPT-1 Stimulating Extracts and Compositions

One aspect of the present invention relates to compositions for topical application which comprise starfruit extracts that stimulate CPT-1 to treat, ameliorate, inhibit, delay, reduce the incidence or risk of, and/or reduce the signs of excess accumulation and/or production of subcutaneous fat. Improving the appearance of skin affected by cellulite and/or combating signs of unwanted subcutaneous fat may include, without limitation, one or more of the following:
(a) reduction in appearance of cellulite lumpiness and/or unevenness;
(b) reduction in pitting appearance of cellulite upon squeezing;
(c) reduction in extent of area affected by cellulite;
(d) prevention or delay in recurrence of cellulite;
(e) prevention or treatment of acne;
(f) prevention or treatment of oily skin;
(g) reduction in subcutaneous fat deposition and/or accumulation;
(h) improvement in collagen deposition; and
(i) improvement in connective tissue strength.

Improvements may be measured by methods known in the art, including, for example, by consumer panel testing. Methods of improving the appearance of skin according to the invention include reducing the appearance of cellulite in skin affected thereby. Methods according to the invention also include improving the tautness or tone of skin affected by cellulite. In practice, the compositions of the invention are applied to skin in need of treatment, that is, skin which suffers from a deficiency or loss in any of the foregoing skin attributes or which would otherwise benefit from improvement in any of the foregoing skin attributes.

A “CPT-1 stimulator” refers to any agent that can decrease the level of triglycerides in human adipocytes via one or more pathways mediated by CPT-1. In some embodiments, the CPT-1 stimulator reduces serum triglycerides. Decrease in triglyceride levels can refer to a decrease in adipocyte proliferation and/or differentiation and/or intracellular lipid and/or triglyceride production, storage, and/or accumulation in adipocytes, and/or an increase in fatty acid oxidation and/or degradation and/or lipolysis; and/or reduced expression of lipogenic genes, in vitro or in vivo, and can be measured by any means known in the art, or as described herein. For example, CPT-1 stimulating starfruit extract can act to decrease triglyceride production within human adipocytes (see, e.g., Example 1 below), or the CPT-1 stimulating starfruit extract can act to decrease serum triglycerides. In some embodiments, triglyceride levels are decreased by at least about 30%, at least about 40%, at least about 50%, at least about 60%, at least about 70%, at least about 75%, or at least about 100%, compared to the level of triglyceride in the absence of the starfruit extract. As another example, CPT-1 stimulation also can be directly measured, e.g., by measuring an increase in CPT-1 gene expression, where the CPT-1 stimulator acts to increase CPT-1 gene expression within human adipocytes. See, e.g., Example 2 below. In some embodiments, CPT-1 gene expression is increased by at least about 10%, at least about 20%, at least about 25%, at least about 30%, at least about 35%, at least about 40%, at least about 50%, or at least about 100%, compared to the level of CPT-1 gene expression in the absence of the CPT-1 stimulating starfruit extract.

It is understood that a CPT-1 stimulator may bring about an effective decrease in triglyceride levels by any means, e.g., by increasing CPT-1 mRNA transcribed and/or increasing CPT-1 protein expressed, and/or decreasing post-translational processing of CPT-1 protein in the adipocytes. Mechanisms of activation can include up-regulating an agonist of CPT-1; down-regulating an antagonist of CPT-1; increasing the stability of CPT-1 RNA and/or protein, and/or increasing dimerization of CPT-1 with ligands that effect CPT-1 activation. The CPT-1 stimulator can refer to an extract, e.g., an extract of Averrhoa carambola, that contains a number of active compounds, one or more of which modulate CPT-1. In a preferred embodiment, the CPT-1 stimulator is a starfruit extract and increases the amount of CPT-1 protein.

In preferred embodiments, the CPT-1 stimulator is an Averrhoa carambola (starfruit) extract, more particularly an aqueous extract. Averrhoa carambola is known also as Carambola and by the common name of “starfruit.” Averrhoa carambola is a species of woody plant in the family Oxalidaceae. It originated from the Philippines, where it is called “balimbing” or “saranate,’ depending on its sourness. Another common name is “belimbing” (Indonesia, Malaysia, India and Sri Lanka). The tree and its fruit are popular throughout Southeast Asia, the South Pacific, parts of East Asia, and it is also cultivated throughout the tropics, such as in Costa Rica, Peru, Colombia, Trinidad, Ecuador, Guyana, Dominican Republic and Brazil, and, in the United States, in south Florida and Hawaii. Averrhoa carambola is a small tree or shrub, with rose to red-purple flowers. The flowers are small and bell-shaped, with five petals that have whitish edges. The flowers are often produced year round under tropical conditions. The tree is cultivated in tropical and semitropical regions for its edible fruits and for medicinal uses. Averrhoa carambola is thought to be rich in antioxidants and vitamin C, and low in sugar, sodium and acids. It is also a potent source of both primary and secondary polyphenolic antioxidants. Averrhoa carambola has both antioxidant and antimicrobial activities: scavenging of NO by the fruit extract is dependent on concentration and stage of ripening. Extracts have antimicrobial activity against E. coli, Salmonella typhi, Staphylococcus aureus and Bacillus cereus.

For use in the compositions of this disclosure, the plant, plant components, and/or active ingredients are preferably derived directly from the plant. The components may be in a pure form, a semi-pure form, or unpurified form. In preferred embodiments, the extract of Averrhoa carambola comprises an aqueous, alcoholic, or hydroalcoholic extract. In more preferred embodiments, components are in the form of an extract obtained by polar solvent extraction, such as by using aqueous extraction.

Solvent extraction involves collecting the raw materials from the plant that contain the desired constituent(s), particularly above-ground parts, such as leaves, stems, flowers, bark, and the like. These plant materials are ground to small particle sizes, and then put into an extracting machine through an inlet for the raw materials by a measurable charging machine. The plant raw material is pushed in the extracting machine by a thruster, and slowly moves the plant raw material forward. Solvent may be added into the machine through a solvent inlet at the top of a waste discharge outlet. Due to the difference in gravity and equilibrium, the solvent flows toward the raw material inlet, soaks the materials, and flows out from the opposite side of the solvent inlet. Since the plant materials and the solvent move in opposite directions against each other, the plant materials are constantly immersed in a solution that contains a low-concentration of extract. As a result of equilibrium, high yield of plant constituent(s) may be achieved by continuously extracting the plant material against the low-concentration solution. The concentration of the plant material in a solvent extraction may be from about 5 g/L to about 50 g/L, preferably from about 10 g/L to about 30 g/L, most preferably at least 20 g/L.

Extraction time can be adapted to remove the plant constituents, for example between about 1-8 hours is typical, more preferably being between about 2-6 hours, and most preferably being between about 3-5 hours. The temperature of extraction can be chosen according to the solvent and the extraction method being used, but may between about 20° C. to about 90° C. (including room temperature extraction), between about 40° C. to about 70° C., or between about 50° C. to about 60° C. The collected extract is then fine-filtered to remove debris, and may be used directly, or may be concentrated, for example by distilling the solvent, by lyophilization, or by other conventional processing. The extract also can be provided in powder form by removal of substantially all of the solvent.

Briefly, a polar or aqueous solvent extraction method involves washing and extracting the plant material using water, an aqueous solution, or other polar solvent. Non-limiting examples of polar solvents include, but are not limited to, water; alcohols (such as methanol, ethanol, propanol, butanol and the like); glycols; ethers (such as diethyl ether, dipropyl ether, and the like); esters (such as butyl acetate, ethyl acetate, and the like); ketones (such as acetone, ethyl methyl ketone, and the like); organic acids including acetic acid, and the like; dimethyl sulfoxide; acetonitrile; other organic solvents; and combinations thereof. Other suitable solvents include physiological saline, phosphoric acid buffer and phosphate buffer saline and the like. In some preferred embodiments, water is used as the polar solvent. Well-known methods in the art may be used for polar solvent extraction.

Similarly, an organic solvent extraction method involves washing and extracting the plant material using an organic solvent. Non-limiting examples of organic solvents include methanol, ethanol, isopropanol, dichloromethane, chloroform, hexane, xylene, and petroleum ether. Well-known methods in the art may be used for organic solvent extraction.

In some embodiments, the extraction comprises aqueous-organic extraction. Generally, aqueous-organic solvent extraction involves initially collecting raw materials from parts of the plant, particularly above-ground parts, such as leaves, stems, flowers, bark, and the like, which are ground into small particle sizes. The ground plant material is soaked in aqueous solution that is acidic or alkaline, depending on the solubility and stability of the desired extract under acidic or alkaline (basic) conditions. For extraction under acidic conditions, an acid such as hydrochloric acid or sulfuric acid is added to water, e.g., at a concentration of about 3% (w/v). For extraction under alkaline conditions, an alkali such as sodium hydroxide or sodium carbonate is added to water. The extraction time and temperature of extraction are typically similar to that used in the organic solvent extraction method described above.

The extract is then collected and fine-filtered to remove debris. Alkaline agents (e.g., ammonia) or acidifying agents (e.g., sulfuric acid) may be added to the extract to neutralize the solution by adjusting the pH, depending on the acidity or alkalinity of the collected extract. The aqueous extract may be used directly, concentrated, or dried.

Alternatively, organic solvent may be added to the neutralized solution to transfer the extract actives from an aqueous phase to an organic phase. Examples of such organic solvents include, but are not limited to, ethanol, isopropanol, butanol, pentanol, hexanol, and xylene. The extract comprising the transferred extract actives dissolved in organic solvent may be used directly, used as a concentrate, or dried.

Different plants containing different constituents may be mixed and extracted together. This process of mixed extraction preferably is used if extracting two or more plants containing constituents having similar solubility in the solvent used for extraction, such as ethanol. The mixture of extracts may be concentrated and stored in an appropriate solvent.

In some embodiments, the extract is obtained from the leaves, stems, and flowers of the Averrhoa carambola plants. In some preferred embodiments, the extracts are obtained by drying the plant material and subsequently extracting from the dried plant using a solvent. For example, a polar solvent may be used. Suitable polar solvents include, but are not limited to, water; alcohols (such as methanol, ethanol, propanol, butanol and the like); glycols; ethers (such as diethyl ether, dipropyl ether, and the like); esters (such as butyl acetate, ethyl acetate, and the like); ketones (such as acetone, ethyl methyl ketone, and the like); organic acids including acetic acid, and the like; dimethyl sulfoxide; acetonitrile; other organic solvents; and combinations thereof. Other suitable solvents include physiological saline, phosphoric acid buffer and phosphate buffer saline and the like. In some preferred embodiments, ethanol and/or water is used as the polar solvent.

Preferably, the Averrhoa carambola extract is obtained by extracting Averrhoa carambola leaves, flowers, and stems with water, ethanol, or a mixture thereof. The solvent systems may optionally comprise from about 10% by volume to about 90% by volume of ethanol, and from about 10% by volume to about 90% by volume of water; from about 25% by volume to about 75% by volume of ethanol and from about 25% by volume to about 75% by volume of water; from about 45% by volume to about 55% by volume of ethanol and from about 45% by volume to about 55% by volume of water; or with a 50:50 mixture (by volume) of ethanol and water.

Preferably, the Averrhoa carambola extract is obtained by solubilization of powdered Averrhoa carambola leaves, followed by enzymatic hydrolysis (for example, by a carbohydrase). The enzymatic activity may be inactivated by heat treatment. An adjuvant is preferably added to remove phenolic compounds, so that the amount of phenolic compounds is less than 2%, less than 1%, or most preferably less than 0.1% of the dry weight of the final Averrhoa carambola extract. The resulting Averrhoa carambola extract preferably contains a total sugar content of between about 20% and 64% with respect to the weight of the dry matter, preferably at least 20% total sugar content with respect to the weight of the dry matter. Alternatively, the total sugar content is from about 3.2 g/L to about 36 g/L, preferably between about 8 g/L and about 16 g/L, compared to the volume of the resulting active extract. Other preferred characteristics of the Averrhoa carambola extract are: dry matter of between about 10 and about 90 g/L, preferably between about 25 and about 40 g/L; a pH from between about 3.0 and about 5.0, preferably from between about 3.0 and about 4.0; protein content from about 8.5 to about 23% by weight of the dry matter; a crude ash content of from about 22.5 to about 39% by weight percentage of the dry matter in the extract; and uronic acid of from about 5 to about 17% by weight of the dry matter in the extract. Particularly preferred Averrhoa carambola leaf extracts according to the invention have at least 20% carbohydrates by weight and no polyphenolic compounds, or substantially undetectable polyphenolic compounds.

Additional suitable extraction processes are disclosed in PCT Publications WO 03/079816 (describing a process for the preparation of tomato extracts), WO 04/014404 (describing a process for the preparation of an Echinacea angustifolia extract) and WO 04/014958 (describing extracting a polysaccharide from Echinacea angustifolia roots), all of which are herein incorporated by reference in their entirety.

In other embodiments, the Averrhoa carambola extract, as referred to herein, includes “synthetic” extracts, i.e., where various combinations of known plant components and/or constituents are combined to substantially mimic the composition and/or activity of a plant extract of natural origin. Such synthetic extracts are included in the terms “extract” or “plant extract.” The synthetic extracts will have two or more, three or more, or four or more active ingredients in common with a natural plant. Most preferably, the synthetic extracts will have substantially the same number of active ingredients as a natural extract of the plant. The correspondence of the numerical incidence of active ingredients between the synthetic extracts and the plant or a natural plant extract may be described in terms of “percent commonality.” Preferably, the synthetic extract has about 50% or more commonality to the chemical composition of a plant or natural plant extract. In other words, the synthetic extract has about 50% or more of the active ingredients found in the plant or a natural plant extract. More preferably, the chemical composition of the synthetic extract has about 70% or more commonality to the chemical composition of a plant or a natural plant extract. Optimally, a synthetic extract has about 90% or more commonality to the chemical composition of a plant or a natural plant extract. The plant or natural plant extract for comparison is derived, for example, from the Averrhoa carambola plant, e.g., as described herein.

The Averrhoa carambola plant may be in any form including, but not limited to, the whole plant, a dried plant, a ground plant, or parts thereof, including but not limited to, seeds, needles, leaves, roots, bark, cones, stems, rhizomes, callus cells, protoplasts, organs and organ systems, and meristems, an extract, a dried extract, a synthetic extract, or components and/or constituents found in, or isolated from, the plant, and/or portions of the plant, or extracts derived either directly or synthetically from the plant, or any combinations thereof.

Substances found in some extracts Averrhoa carambola include derivatives of abscisic acid and abscisic alcohol, vomifoliol, roseoside, epicatechin, 2,5-dimethoxy-3-undecylphenol and 5-methoxy-3-undecylphenol, 5-O-methylembelin, and 2-dehydroxy-5-O-methylembelin, among others (Araho, D. et al., Nat. Med. 59:113-16, 2005; Xu et al., 2004; Chakthong, et al., Chinese Chem. Lett. 21(9):1094-96, 2010, D.C. Gunawardena, Proc. Peradeniya Univ. Res. Sessions, 2007). Some embodiments may include one or more these substances, while others may be free, or substantially free, of one or more of these substances.

Cosmetic compositions of the instant invention generally comprise an amount of a CPT-1 stimulator, e.g., an amount of Averrhoa carambola extract, effective to improve the appearance to human skin in an area to which it is topically applied. In preferred embodiments, the compositions comprise an amount of starfruit extract effective to decrease adipocyte differentiation and/or intracellular triglyceride production and/or accumulation in adipocytes in the area of skin.

In certain preferred embodiments, the cosmetic composition comprises an amount of starfruit extract from about 0.001 weight % to about 50 weight % based on the total weight of the composition; preferably from about 0.01 weight % to about 25 weight % based on the total weight of the composition; more preferably from about 0.1 weight % to about 10 weight % based on the total weight of the composition, and even more preferably from about 0.1 weight % to about 1 weight %, or about 0.5 weight %, based on the total weight of the composition. In one embodiment, a cosmetic composition comprises an amount of starfruit extract from about 0.01 weight % to about 5 weight % based on the total weight of the composition. The above amounts refer to an “active amount” of a CPT-1 stimulator, such as the amount of starfruit or Averrhoa carambola extract. The term “active amount” refers to the amount of starfruit equivalent to the concentration of dry absent diluent, solvent, carrier, filler or the like. Cosmetic compositions described herein find use as anti-lipid agents, e.g., as detailed below.

Cosmetic Use of CPT-1 Modulating Compositions

Another aspect of the instant invention relates to cosmetic use of compositions comprising a CPT-1 stimulator, particularly an aqueous Averrhoa carambola extract, where the starfruit extract stimulates CPT-1 expression. Such compositions act to ameliorate, inhibit, delay, reduce, and/or improve the signs of excess accumulation and/or production of subcutaneous fat, and accordingly find use as potent anti-lipid products, and in particular anti-cellulite products.

An “anti-lipid” agent or product, as used herein, refers to any substance that acts to decrease triglyceride levels in adipocytes, such as by bringing about one of more of a decrease in adipocyte proliferation and/or adipocyte differentiation; a decrease in intracellular lipid and/or triglyceride production, storage, and/or accumulation, an increase in fatty acid oxidation, degradation and/or lipolysis; and/or reduced expression of lipogenic genes, in vitro or in vivo. An “anti-cellulite” agent is product, as used herein, is a substance that exerts in anti-lipid effects so as to produce a visible or palpable improvement in skin affected by cellulite.

In some embodiments, a method is provided for improving the appearance of skin affected by subcutaneous fat production and/or accumulation, such as in the case of cellulite, where the method comprises topically applying to affected skin at least one CPT-1 stimulator such as starfruit extract, in a cosmetically acceptable vehicle. The composition will comprise an effective amount of the substance. An “amount effective” or an “effective amount” to improve appearance to the skin refers to the active amount of a CPT-1 stimulator such as a starfruit extract sufficient to provide a visible improvement in skin affected by unwanted subcutaneous fat when applied to the skin for a sufficient time. Such improvements include without limitation, the following:
(a) reduction in appearance of cellulite lumpiness and/or unevenness;
(b) reduction in pitting appearance of cellulite upon squeezing;
(c) reduction in extent of area affected by cellulite;
(d) prevention or delay in recurrence of cellulite;
(e) prevention or treatment of acne;
(f) prevention or treatment of oily skin;
(g) reduction in subcutaneous fat deposition and/or accumulation;
(h) improvement in collagen deposition; and/or
(i) improvement in connective tissue strength.

The compositions of the invention can be applied to skin in need of treatment, such as skin which suffers from a deficiency or loss in any of the foregoing attributes or which would otherwise benefit from the composition's anti-lipid effects, e.g., as described herein. For example, the CPT-1 stimulator, such as an Averrhoa carambola aqueous leaf extract, can be provided in a cosmetically acceptable vehicle, topically applied to a desired area of skin, and allowed to remain on the area in an amount effective to treat and/or prevent an unwanted feature or condition of the skin, and/or to improve the aesthetic appearance of the skin. Topical application facilitates targeted delivery of the active components, e.g., without the requirement of an injection or the expertise of a health practitioner. While topical compositions are a preferred embodiment according to the invention, oral formulations are also contemplated.

“Treatment” as used herein, as well as related terms such as “treat” or “treating,” refers to eradicating, reducing, ameliorating, or reversing one or more of the unwanted features associated with the skin condition being treated, such that the consumer perceives an improvement in the appearance of the skin or other treatment benefit with respect to the condition. “Prevention” as used herein, as well as related terms such as “prevent” or “preventing,” refers to affording skin not yet affected by the condition a benefit that serves to avoid, delay, forestall, minimize, or reduce the recurrence one or more unwanted features associated with the skin condition to be prevented. Such preventative benefits include, for example, delaying development and/or recurrence of the condition, or reducing the duration, severity, or intensity of one or more unwanted features associated with the condition if it eventually develops.

Methods of Use of the Compositions

Cosmetic compositions taught herein, according to methods of the invention, can be applied to an area of skin affected by cellulite to improve the appearance of the skin. An improvement may involve a reduction in appearance of lumpiness and/or unevenness, characteristic of cellulite, preferably reducing what is known as the “cottage cheese” or “orange peel” look. Further, areas of cellulite tend to bulge, pit, and dimple when squeezed or compressed, as occurs when legs are crossed when seated, which can worsen the appearance of cellulite areas. In some embodiments, an improvement involves a reduction in this pitting appearance of cellulite upon squeezing, so that the look of cellulite on the legs appears reduced even when sitting with the legs crossed. An improvement may also involve reducing the visible depth and/or intensity of cellulite.

Cellulite tends to accumulate on certain body regions, e.g., on the thighs and buttocks of many women, as well as on the abdomen, hip and/or upper arm region. In some embodiments, the extent of the area affected by cellulite is reduced, such that smaller areas of the thigh, buttocks, abdomen, hip, and/or upper arm region remain visibly affected. In preferred embodiments, one of more such regions becomes free of visible signs of cellulite following treatment with a composition described herein.

In some embodiments, a method is provided for reducing the re-occurrence of cellulite in an area that was previously affected by cellulite, but showing little or no signs of cellulite. Reducing the re-occurrence refers to delaying the recurrence of any cellulite on a previously-affected area, or reducing the extent of cellulite that re-appears on the area, such that any recurrent cellulite is less noticeable than previous amounts.

Compositions for use in the method of the instant invention will comprise a CPT-1 stimulator, such as starfruit extract, in an amount sufficient to reduce intracellular triglyceride levels in adipocites at a given area of skin when topically applied thereto. As used herein, reducing triglyceride levels and related expressions refer to a decrease in adipocyte differentiation and/or intracellular triglyceride production, storage, and/or accumulation in adipocytes, and/or an increase in fatty acid oxidation; and/or reduced expression of lipogenic genes, in vitro or in vivo, to decrease the triglyceride content in an area of skin, preferably improving skin appearance to a perceptible extent. For example, in some embodiments, the triglyceride level is decreased by at least about at least about 30%, at least about 40%, at least about 50%, at least about 60%, at least about 70%, at least about 75%, or at least about 100%, compared to the level of triglycerides in the absence of the CPT-1 stimulating starfruit extract. Triglyceride levels in subcutaneous adipocytes can be determined by appropriate assays, e.g., in vitro assays described herein or known in the art. For example, Example 1 below provides experimental details of assays for measuring intracellular triglyceride levels in human adipocytes.

Without wishing to be bound by theory, compositions disclosed herein act by a number of mechanisms of action to effect improvement in the appearance of skin affected by unwanted subcutaneous fat. The compositions comprising starfruit extracts act as CPT-1 stimulators. The Carnitine Palmitoyl Transferase-1 enzyme (CPT-1), also known as carnitine acyl transferase I, or CAT-1, is a mitochondrial enzyme. It is part of a family of enzymes called carnitine acyltransferases. Three isoforms of CPT-1 are currently known: CPT1A, CPT1B, and CPT1C. CPT-1 is associated with the outer mitochondrial membrane and mediates the transport of long-chain fatty acids across the membrane by binding them to carnitine. Its role in fatty acid metabolism makes CPT-1 important in many metabolic disorders such as type 2 diabetes and insulin resistance. Such diseases, along with many other health problems, cause free fatty acid (FFA) levels in humans to become elevated, fat to accumulate in skeletal muscle, and a decrease in the ability of muscles to oxidize fatty acids. CPT-1 has been implicated in playing a critical role in these symptoms. Its importance in fatty acid metabolism makes CPT-1 a potentially useful enzyme to focus on in the development of treatments of many other metabolic disorders as well. The CPT-1 stimulating starfruit extract may act to break up fatty deposits, even in mature fat cells. Furthermore, this extract can be beneficial in treating other skin conditions characterized by excess lipids, e.g., acne or oily skin.

Thus, without wishing to be bound by theory, compositions disclosed herein may act to combat signs of cellulite via more than one mechanism of action. The CPT-1 stimulating starfruit extracts work to decrease subcutaneous fat deposition and/or accumulation and/or decrease adipocyte differentiation. CPT-1 is an adipocyte differentiation marker, and it acts to reduce adipocyte differentiation. A stronger dermal structure reduces the likelihood of fat nodules “blebbing” between connective tissue fibers or septa, which is believed to lead to the characteristic unsightly appearance of cellulite. Further, lower levels of subcutaneous fat further reduce the likelihood of such blebbing. As cellulite is believed to result from a combination of enlarged fat tissue and weak dermal structure, combating cellulite through multiple approaches, as described herein, can provide superior results compared with products that utilize only one approach. Accordingly, the invention provides novel mechanisms of action to improve the appearance of cellulite, and thus potent anti-cellulite compositions for use therein.

In some embodiments, the cosmetic compositions for combating signs of unwanted subcutaneous fat can further comprise additional anti-lipid agents. For example, the cosmetic composition comprising a CPT-1 stimulating starfruit extract in an amount effective (or amounts effective) to improve the appearance of skin may further comprise at least one other anti-lipid agent, including one other anti-cellulite agent. It is contemplated that synergistic improvements may be obtained with such combinations, in some embodiments.

Exemplary anti-cellulite agents include, without limitation, phosphodiesterase inhibitors, such as xanthine analogs, caffeine, aminophylline, and theophylline; adenylate cyclase activators, such as forskolin and Coleus forskohlii extract; lipolysis stimulators, such as hawthorne extract and cola extract; beta adrenergic receptor agonists, such as isoproterenol; alpha-2-adrenergic antagonists, such as yohimbine and Ginkgo biloba extract; perilla oil (see, e.g., U.S. Pat. No. 7,410,658); carnitine and/or creatine (see, e.g., US 2007/0264205 entitled “Cosmetic Composition having Carnitine Creatinate and Methods for Using,” incorporated herein by reference in its entirety). In some embodiments, additional actives may include a collagen stimulator and/or an elastin stimulator, e.g., a substance that stimulates elastin production, and/or a glycosaminoglycan enhancer. Examples of collagen, elastin and glycosaminoglycan enhancers include, e.g., fennel extract, carrot extract, and alfalfa extract. In some embodiments, the additional actives may include a collagenase inhibitor and/or elastase inhibitor. In some embodiments, the invention relates to synergistic action of one or more compositions described herein with perilla oil, e.g., to provide enhanced anti-cellulite benefits to skin.

In some embodiments, the cosmetic compositions can further comprise at least one collagen and/or elastin stimulator. Such collagen or elastin stimulators are effective in, for example, providing improvement in procollagen and/or collagen production and/or improvement in maintenance and remodeling of elastin.

Anti-cellulite and Adiposity Benefits

In some embodiments, a method is provided for reducing obesity and/or increasing weight loss and/or aiding body sculpting. The method can comprise topically applying to an area affected by unwanted fat deposition an effective amount of a CPT-1 stimulating Averrhoa carambola extract, in a cosmetically acceptable vehicle, for a time sufficient to reduce the unwanted fat. The CPT-1 stimulating activities of the composition can reduce fat accumulation and/or adipocyte differentiation, as described herein, to reduce weight, preferably in targeted areas. Such areas may be “problem areas” from which the consumer finds it difficult to loose weight by general dieting and/or exercise. Other approaches for treating unwanted fat deposition have been described and may be used with the CPT-1 stimulating Averrhoa carambola extract disclosed herein. See, e.g., WO 04/047746.

In some other embodiments, it is contemplated that compositions described herein, such as cosmetic compositions comprising an Averrhoa carambola extract, will exhibit one or more benefits on aesthetic appearance, selected from the following:

(a) treatment, reduction, and/or prevention of fine lines or wrinkles,
(b) reduction of skin pore size,
(c) improvement in skin thickness, plumpness, and/or tautness;
(d) improvement in skin suppleness and/or softness;
(e) improvement in skin tone, radiance, and/or clarity;
(f) improvement in procollagen and/or collagen production;
(g) improvement in maintenance and remodeling of collagen and/or elastin;
(h) improvement in skin texture and/or promotion of retexturization;
(i) improvement in skin barrier repair and/or function;
(j) improvement in appearance of skin contours;
(k) restoration of skin luster and/or brightness;
(l) replenishment of essential nutrients and/or constituents in the skin;
(m) decreased by aging and/or menopause;
(n) improvement in skin moisturization;
(o) increase in skin elasticity and/or resiliency;
(p) treatment, reduction, and/or prevention of skin sagging; and/or
(q) reduction of pigment spots.

Based on the teachings provided herein, one of skill in the art will recognize other cosmetic and/or pharmaceutical applications for the compositions described herein, and such applications are also contemplated as within the scope of the instant invention. For example, compositions described herein may also find use in personal care products, such as skin care or hair care products, where it is desirable to produce an improvement in the appearance of skin or of hair, as described herein, upon application of the product. It is contemplated, for example, that compositions described herein can find use in lotion and/or tonic formulations that decrease the appearance of cellulite and other unwanted subcutaneous fat on various surfaces of the body. It is contemplated, for example, that compositions described herein can find use in hair care formulations which improve the appearance of hair by decreasing sebum and/or oil and/or unwanted greasiness on the hair.

Personal care products for the skin according to the invention include, for example, body lotions, body tonics, and the like. Hair care products according to the invention include, for example, shampoo, conditioner, aerosol spray, pump spray, mousse, foam, solution, serum, or the hair care composition may be incorporated into a towelette.

Treatment Regimens

The invention provides methods for improving the appearance of skin by topically applying a composition comprising a CPT-1 stimulating starfruit extract containing composition over an area of skin for a period of time sufficient to improve the appearance of skin, as described herein. The composition will typically be applied to the skin in accordance with a treatment regime. The treatment regimen can comprise application one, two, or three times daily for as long as is necessary to achieve desired results, such as the anti-cellulite benefits described herein. This treatment regimen may comprise daily application or every-other-day application for at least about one week, at least about two weeks, at least about three weeks, at least about four weeks, at least about six weeks, at least about eight weeks, at least about twelve weeks, or more. In some embodiments, the composition is applied more than once daily for the recited periods of time, for example, twice daily, preferably once in the morning and once again at night before bed. The composition preferably is massaged thoroughly onto the area to be treated, e.g., onto the thighs, buttocks, hips, abdomen, upper arms, and the like.

Chronic treatment regimens are also contemplated, e.g., with respect to prophylactic treatments aimed at forestalling one or more signs of skin cellulite or other unwanted subcutaneous fat; as well as with respect to reducing and/or preventing the recurrence of cellulite in an area previously affected thereby. The treatment and/or prophylactic regime may also depend on concentration of the CPT-1 stimulating starfruit extract being used, e.g., as different concentrations may produce anti-cellulite skin benefits more quickly than others. The treatment regimens according to the invention may optionally include additional exercise, diet modulation and increased water intake.

The compositions generally are topically applied to the skin for a period of time sufficient to improve the appearance of skin affected by cellulite or other unwanted subcutaneous fat. In some embodiments, the compositions are left on the skin as a “leave-on” composition, by which is meant they are applied in a formulation that is allowed to remain in the skin without being deliberately washed and/or rubbed off for a certain period of time. For example, the composition may be left on the skin for a day, overnight, or for at least about 18 hours, for at least about 12 hours, for at least about 8 hours, or for at least about 4 hours.

Formulations of Averrhoa carambola Extracts

CPT-1 stimulators, such as aqueous extracts of the leaf of Averrhoa carambola, may be used to formulate cosmetic compositions, as known in the art. The cosmetic compositions find use in anti-cellulite and anti-lipid products, preferably formulated for topical application to the skin e.g., with a cosmetically acceptable vehicle. Formulations for cosmetic compositions comprising CPT-1 stimulating Averrhoa carambola extracts are described in more detail below.

In accordance with the invention, the CPT-1 stimulating Averrhoa carambola extracts may be formulated in a variety of product forms. The compositions may be prepared in targeted delivery systems, e.g., creams, lotions, gels, toners, serums, transdermal patches, and the like, particularly for topical administration. For example, the invention encompasses compositions comprising a cosmetically or dermatologically acceptable formulation which is suitable for contact with living animal tissue, particularly human tissue, with virtually no or little adverse physiological effect to the user. The invention also encompasses compositions for oral delivery. Compositions embraced by this invention can be provided in any cosmetically and/or dermatologically suitable form, preferably as a lotion or cream, but also in an anhydrous or aqueous base, as well as in a sprayable liquid form. Other suitable cosmetic product forms for the compositions include, for example, an emulsion, a cream, a balm, a gloss, a lotion, a mask, a serum, a toner, an ointment, a mousse, a patch, a pomade, a solution, a spray, a wax-based stick, a towelette, a shampoo, a conditioner and/or a foam

In some particular embodiments, the cosmetic composition comprising a CPT-1 stimulating Averrhoa carambola aqueous extract is provided in the form of a cream for topical application to skin affected, previously-affected, or likely-to-be affected by cellulite. In some particularly preferred embodiments, the cream comprising the CPT-1 stimulating aqueous starfruit extract is supplied along with a gel for use with the cream, for example, by following application of the cream with application of the gel to the same area of skin. The gel preferably provides tightening polymers to enhance the cellulite-reducing effects of the cream. In more preferred embodiments, the gel provides a cooling sensation to the skin when applied to the skin following application of the cream. The cream and gel may be provided in different containers, or in different compartments of the same container. In some embodiments, the cream and gel are provided in a “tube-within-a-tube” that dispenses the cream and gel together. This allows the cream and gel to be mixed upon dispensing, e.g., immediately before application to the skin.

In addition, the compositions contemplated may include one or more compatible cosmetically acceptable adjuvants commonly used and known by the skilled practitioner, such as colorants, fragrances, emollients, humectants, preservatives, vitamins, chelators, thickeners, perilla oil or perilla seed oil (WO 01/66067 to a “Method of Treating a Skin Condition,” incorporated herein by reference) and the like, as well as other botanicals such as aloe, chamomile, and the like.

Also embraced by the invention are transdermal modes of delivery, such as patches and the like, with or without suitable penetration enhancers. The methods and compositions embodied by the invention provide a means by which the CPT-1 stimulating Averrhoa carambola extract can be effectively administered in a transdermal system or device. Examples of such devices are known in the art, e.g., as disclosed in U.S. Pat. Nos. 5,146,846; 5,223,262; 4,820,724; 4,379,454; and 4,956,171, all of which are incorporated herein by reference and such descriptions are not meant to be limiting. In a preferred method, topical application is through a sustained release vehicle, carrier, or diluent, e.g., using a topically applied sustained release patch. Preferably, when a topical patch is used, the patch is applied to the desired area for extended period of time, such as, e.g., at least about 4 hours, at least about 8 hours, at least about 12 hours, at least about 16 hours, or at least about 24 hours. In some embodiments, the extended period of time is all day, e.g., from the morning to bedtime, or overnight, e.g., while the user is sleeping.

The CPT-1 stimulating aqueous extracts of the leaf of Averrhoa carambola of the present invention are preferably contained in a cosmetically or dematologically acceptable vehicle, medium, diluent or carrier, providing a topical formulation for use in treating, ameliorating, preventing, inhibiting, delaying, and/or reducing the signs of excess accumulation and/or production of subcutaneous fat, including improving the appearance of skin affected by cellulite.

In some embodiments, the topical formulation comprises a cosmetically acceptable vehicle (medium, diluent, or carrier) that is compatible with human skin. The cosmetically acceptable vehicle may comprise an aqueous phase, an oil phase, alcohol, or aqueous/alcohol-based solutions, ointments, lotions, gels, wax-in-water emulsions, or water-in-oil, oil-in-water, or water-oil-water emulsions, e.g., having the appearance of creams, gels, microemulsions, or aerosols.

The aqueous phase is a mixture of one or more water soluble or water dispersible substances, which can be liquid, semi-solid or solid at room temperature (25° C.). The vehicle comprises, or can be in the form of, a suspension, dispersion, or solution in water or in an aqueous-alcoholic vehicle, which may contain a thickener or gellant. A person skilled in the art can select the appropriate cosmetic form, the ingredients contained therein, as well as the method for preparing it, on the basis of the knowledge that the skilled artisan possesses.

In some embodiments, the cosmetically acceptable vehicle may include an aqueous phase which may contain water, or a mixture of water and at least one hydrophilic organic solvent, in particular an alcohol, especially a linear or branched lower monoalcohol containing from 2 to 5 carbon atoms, e.g., ethanol or propanol; a polyol, e.g., propylene glycol, sorbitol, glycerol, diglycerol, panthenol, or polyethylene glycol; and mixtures thereof. This aqueous phase may represent from about 0.5 weight % to about 99.99 weight %, based upon the total weight of the composition.

In some embodiments, when the composition of the invention is in the form of an emulsion, the composition may also optionally comprise a surfactant, preferably in an amount from about 0.1 weight % to about 30 weight %, and in particular, from about 1 weight % to about 20 weight %, based upon the total weight of the composition.

In some embodiments, the composition may also comprise a thickening polymer such as an amphiphilic polyurethane, a polyacrylic homopolymer or copolymer, a polyester, and/or a hydrocarbon-based resin.

The invention also contemplates formulations that may comprise an oil phase containing oil-soluble or oil-dispersible substances, which are liquid at room temperature (25° C.) and/or oily or waxy substances that are solid at room temperature, such as waxes, semi-solids, gums, and mixtures thereof. The waxes can include hydrocarbon-based waxes, fluoro waxes and/or silicone waxes and can be of plant, mineral, animal, and/or synthetic origin. Formulations typically comprise from about 0 weight % to about 20 weight % waxes, based upon total weight. The gums used are generally high molecular weight cyclic polydimethylsiloxanes (PDMS), cellulose gums or polysaccharides, and the semi-solid materials are generally hydrocarbon-based compounds, such as, but not limited to, lanolins and derivatives thereof. This oily phase may also contain organic solvents.

Suitable oily materials that are liquid at room temperature, often referred to as oils, include hydrocarbon-based oils of animal origin such as perhydrosqualene; hydrocarbon-based plant oils such as liquid triglycerides of fatty acids of 4 to 10 carbon atoms, for instance, heptanoic or octanoic acid triglycerides, or oils such as sunflower oil, corn oil, soybean oil, grapeseed oil, castor oil, avocado oil, caprylic/capric acid triglycerides, or jojoba oil; linear or branched hydrocarbons of mineral or synthetic origin, such as liquid paraffins and derivatives thereof, or petroleum jelly; synthetic esters and ethers, in particular esters of fatty alcohols, namely, for example, isopropyl myristate, 2-ethylhexyl palmitate, 2-octyldodecyl stearate, isostearyl isostearate; hydroxylated esters such as isostearyl lactate, octyl hydroxystearate, octyldodecyl hydroxystearate, heptanoates, octanoates and decanoates of fatty alcohols; polyol esters such as propylene glycol dioctanoate, neopentyl glycol diheptanoate, diethylene glycol diisononanoate, and pentaerythritol esters; fatty alcohols containing from 12 to 26 carbon atoms such as octyldodecanol, 2-butyloctanol, 2-hexyldecanol, 2-undecylpentadecanol, oleyl alcohol; partially hydrocarbon-based fluoro oils and/or fluorosilicone oils; silicone oils such as volatile or non-volatile, linear or cyclic polydimethylsiloxanes (PDMS) that are liquid or semisolid at room temperature such as cyclomethicones and dimethicones, optionally comprising a phenyl group, for instance phenyl trimethicones, siloxanes, and mixtures thereof. These oils are usually present in an amount of about 0 weight % to about 90 weight %, preferably from about 1 weight % to about 80 weight % by weight of the oil phase.

The oil phase of the formulation may also comprise one or more cosmetically acceptable organic solvents. These solvents are present in an amount of from about 0 weight % to about 60 weight %, preferably from about 1 weight % to about 30 weight %, based on the total weight of the composition, and may be selected from the group consisting of lipophilic organic solvents, amphiphilic organic solvents, and mixtures thereof. Suitable solvents which may be used in the composition of the invention include acetic acid esters such as methyl, ethyl, butyl, amyl or 2-methoxyethyl acetate; isopropyl acetate; hydrocarbons such as toluene, xylene, p-xylene, hexane or heptane; ethers containing at least 3 carbon atoms, and mixtures thereof. In some other embodiments, the compositions can be in the form of vesicular dispersions containing ionic and/or nonionic lipids, as described above.

In yet other embodiments, the compositions are formulated into liposomes or microspheres, which can comprise other additives or substances, and/or which can be modified to more specifically target or remain at a site following administration. (See, e.g., U.S. Pat. No. 5,770,222 to Unger et al., incorporated herein by reference.)

The formulations for use in the inventive methods may further comprise any ingredient conventionally used in the cosmetics field. These ingredients include, e.g., preserving agents, aqueous phase thickeners (polysaccharide biopolymers, synthetic polymers), fatty-phase thickeners, fragrances, hydrophilic and lipophilic active agents, and mixtures thereof. The amounts of these various ingredients are those conventionally used in the cosmetics field to achieve their intended purpose, and range typically from about 0.01 weight % to about 20 weight %, based upon the total weight of the composition or formulation. The nature of these ingredients and their amounts will be selected to be compatible with the production and intended applications of the compositions, as described herein.

In some embodiments, the formulation may optionally comprise an additional particulate phase, typically present in an amount of from about 0 weight % to about 30 weight %, based upon the total weight of the composition or formulation, preferably from about 0.05 weight % to about 20 weight %, and which can comprise pigments and/or pearlescent agents and/or fillers used in cosmetic compositions.

Suitable inorganic pigments include, but are not limited to, titanium oxide, zirconium oxide and cerium oxide, as well as zinc oxide, iron oxide, chromium oxide and ferric blue. Suitable organic pigments include barium, strontium, calcium, and aluminium lakes and carbon black. Suitable pearlescent agents include mica coated with titanium oxide, with iron oxide, or with natural pigment. Fillers are normally present in an amount from about 0 weight % to about 20 weight %, based on the total weight of the composition or formulation, preferably from about 0.1 weight % to about 10 weight %. Suitable fillers include talc, silica, zinc stearate, mica, kaolin, nylon (in particular orgasol) powder, polyethylene powder, TEFLON™, starch, boron nitride, copolymer microspheres such as Expancel (Nobel Industrie), Polytrap (Dow Corning), and silicone resin microbeads (Tospearl from Toshiba).

In some particular embodiments, the compositions for topical application can be in the form of a personal care product for the skin, preferably for the thighs, buttocks, legs, hips, abdomen, limbs, upper arms, and/or other areas of the body. Non-limiting examples include creams or lotions, salves, ointments, gels, masks, artificial tanning compositions, patches, or a solid which is poured or cast as a stick or a dish, for example.

In some embodiments, the topical formulations may also include one or more antioxidants. An antioxidant functions, among other things, to scavenge free radicals from skin, protecting the skin from environmental aggressors. Examples of antioxidants that may be used in the present compositions and formulations include compounds having phenolic hydroxy functions, such as ascorbic acid and its derivatives/esters; thiodipropionic acid and its esters; vitamins A, C, or E; polyphenols, beta-carotene; catechins; curcumin; ferulic acid derivatives (e.g. ethyl ferulate, sodium ferulate); gallic acid derivatives (e.g., propyl gallate); lycopene; reductic acid; rosmarinic acid; tannic acid; tetrahydrocurcumin; tocopherol and its derivatives; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Compositions of the present invention may have an antioxidant preferably from about 0.001 weight % to about 10 weight %, and more preferably from about 0.01 weight % to about 5 weight %, based on the total weight of the composition or formulation.

In some embodiments, the topical formulations may also include one or more of the following: a skin penetration enhancer, an emollient, a skin plumper, an exfoliation promoter, and an optical diffuser. Details with respect to these and other suitable cosmetic ingredients can be found in the International Cosmetic Ingredient Dictionary and Handbook, 10th Edition (2004), published by the Cosmetic, Toiletry, and Fragrance Association (CTFA), at pp. 2177-2299, which is herein incorporated by reference in its entirety.

An emollient provides the functional benefits of enhancing skin smoothness and may aid in improving the appearance of skin affected by cellulite and other unwanted subcutaneous fat. Examples of emollients include isopropyl myristate, petrolatum, isopropyl lanolate, silicones (e.g., methicone, dimethicone), oils, mineral oils, fatty acid esters, or any mixtures thereof. The emollient is preferably present from about 0.1 wt % to about 50 wt % of the total weight of the composition or formulation.

A skin plumper serves as a collagen enhancer to the skin. An example of a suitable, and preferred, skin plumper is palmitoyl oligopeptide. Other skin plumpers are collagen and/or glycosaminoglycan (GAG) enhancing agents. The skin plumper is preferably present from about 0.1 weight % to about 20 weight % of the total weight of the composition or formulation.

In some embodiments, formulations may have one or more exfoliation promoters. Suitable examples of exfoliation promoters include alpha hydroxy acids (AHA); benzoyl peroxide; beta hydroxy acids; keto acids, such as pyruvic acid, 2-oxopropanoic acid, 2-oxobutanoic acid, and 2-oxopentanoic acid; oxa acids as disclosed in U.S. Pat. Nos. 5,847,003 and 5,834,513 (the disclosures of which are incorporated herein by reference); salicylic acid; urea; or any mixtures thereof. The preferred exfoliation promoters are 3,6,9-trioxaundecanedioic acid, glycolic acid, lactic acid, or any mixtures thereof. When an embodiment of the invention includes an exfoliation promoter, the formulation may have from about 0.1 weight % to about 30 weight %, preferably from about 1 weight % to about 15 weight %, and more preferably from about 1 weight % to about 10 weight %, of the exfoliation promoter based on the total weight of the composition or formulation.

An optical diffuser is a particle that changes the surface optometrics of skin, resulting in a visual blurring and softening of, for example, lines and wrinkles, as well as lumpiness and unevenness caused by cellulite and other unwanted subcutaneous fat. Examples of optical diffusers that can be used in the present invention include, but are not limited to, boron nitride, mica, nylon, polymethylmethacrylate (PMMA), polyurethane powder, sericite, silica, silicone powder, talc, TEFLON™, titanium dioxide, zinc oxide, or any mixtures thereof. The optical diffuser is preferably present from about 0.01 weight % to about 20 weight %, based on the total weight of the composition or formulation.

In some embodiments, formulations may have one or more retinoids. Exemplary retinoids include, without limitation, retinoic acid (e.g., all-trans or 13-cis) and derivatives thereof, retinol (Vitamin A) and esters thereof, such as retinol palmitate, retinol acetate and retinol propionate, and salts thereof.

In some embodiments, formulations may have one or more sunscreen protectors. A sunscreen protects the skin from damaging ultraviolet rays. In an illustrative embodiment of the invention, the sunscreen would provide both UVA and UVB protection, by using either a single sunscreen or a combination of sunscreens. Among the sunscreens that can be employed in the present compositions are avobenzone, cinnamic acid derivatives (such as octylmethoxy cinnamate), octyl salicylate, oxybenzone, titanium dioxide, zinc oxide, or any mixtures thereof. The sunscreen may be present in an amount from about 1 weight % to about 30 weight % of the total weight of the composition. The compositions of the invention having sunscreen bring about additional improvements to the aesthetic appearance of skin, including at least one of the following: minimizing sun-burning and/or reducing redness.

In some embodiments, the formulation may also have one or more of the following cosmetic and pharmaceutical active agents, excipients, ingredients, or adjuvants: anesthetics; antibiotics, e.g., erythromycins and tetracyclines; salicylic acids; anti-allergenics; antifungals; antiseptics; anti-irritants; anti-inflammatory agents; antimicrobials; analgesics; nitric oxide synthase inhibitors; insect repellents; self-tanning agents; skin penetration enhancers; skin cooling agents; chelating agents; colorants including dyes, lakes and pigments that may be untreated or chemically surface treated to improve wetability or some other property; demulcents; emulsifiers; fragrances; humectants; lubricants; skin protectants; moisturizers; pH adjusters; preservatives; stabilizers; surfactants; thickeners; film formers; plasticizers; viscosity modifiers; vitamins; blood flow stimulators; or any mixtures thereof. The amounts of these various substances are those that are conventionally used in the cosmetic or pharmaceutical fields to achieve their intended purposes, for example, they may constitute from about 0.01 weight % to about 20 weight % of the total weight of the composition or formulation.

Emulsifiers are typically present in the compositions or formulations of the invention in an amount from about 0.01 weight % to about 30 weight %, and preferably from about 0.5 weight % to about 30 weight %, based on the total weight of the composition. In some other embodiments, the composition or formulation is free or substantially free of emulsifiers.

Non-limiting examples of suitable thickening agents include xanthan gum, hydroxypropyl cellulose, hydroxyethyl cellulose, carbomer, gum acacia, Sepigel 305 (available from Seppic Co., France), and clays such as magnesium aluminum silicate.

The topical compositions of the present invention may include, and their utility can be enhanced, by one or more humectants, such as ureas, pyrrolidone carboxylic acids, amino acids, sodium hyaluronates, certain polyols, and other compounds with hygroscopic properties.

The general activity and mildness to skin of compositions according to the invention can also be enhanced by neutralization to a pH from about 3.5 to about 8.0, most preferably a pH from about 3.5 to about 5.5. This neutralization is preferably accomplished with one or more of ammonium hydroxide, potassium hydroxide, sodium hydroxide, arginine or other amino acids, and/or triethanolamine.

All terms used herein are intended to have their ordinary meaning unless otherwise provided. As used herein, “% by weight” or “% wt” refers to the weight percent of a component in relation to the total weight of the composition or formulation (i.e., including any carriers, vehicles, solvents, emollients, fillers, or other components added before application to the skin) unless otherwise specified.

### EXAMPLES

### Example 1

### Averrhoa carambola Leaf Extraction Procedure and HPLC Analysis of Averrhoa carambola Leaf Extracts from Two Protocols

Averrhoa carambola can be extracted from natural raw materials by using methods of aqueous-organic solvent extraction as is known in the art, e.g., as set forth below.

A CPT-1—stimulating aqueous extract was obtained by extracting the leaves of the Averrhoa carambola plant using an aqueous extraction scheme. Briefly, leaves of Averrhoa carambola were first ground to produce a powder.

The ground powder was then solubilized in water at a concentration of between about 10 and about 90 g/L, preferably between about 25 and about 40 g/L, at a temperature of between about 40° C. and 60° C. The aqueous solution was submitted to enzymatic hydrolysis targeting carbohydrates using any one of a number of commercially available hydrolases or carbohydrases. Residual enzymatic activity was inactivated by heat treatment for approximately 4 hours. An adjuvant such as polyvinylpolypyrrolidone (PVPP) was added to eliminate polyphenolic compounds. The soluble and insoluble phases were separated by decantation, filtration, and/or centrifugation and collection of the soluble fraction (filtrate or soluble phase). Filtration eliminated high molecular weight molecules, i.e., above 20,000 Da. The active fraction was then concentrated by methods known in the art, such as by rotovap, lyophilization, or freeze drying. The resulting concentrated liquid phase was sterilized by membrane filtration through a 0.22 micrometer filter. The total concentrated extract gave an aqueous extract (CPT-1 stimulating extract) of Averrhoa carambola in the form of a clear, amber colored liquid.

The CPT-1 stimulating extract was characterized as having 36.2 g/L dry matter (obtained via oven drying at 105° C. until constant weight was obtained); a pH of 3.6 (measured potentiometrically at room temperature); 56.1% total sugar content by weight of the dry matter (Dubois et al., Analytical Chemistry 28(3):350-356 (1956)); 26.2% crude ash content by weight of the dry matter (obtained via incineration at 550° C. in an electric muffle furnace and deducting the tared container, VULCAN™ 3.550-NDI); 8.6% protein content by weight of the dry matter (measured according to the Kjeldhal method, Official Methods of Analysis of AOAC International, 12^{th} Ed., pages 15-60 (1975)); 6.6% uronic acid by weight of the dry matter, and 0.0015% polyphenol content by weight of the dry matter. Uronic acid determination was made by reacting uronic acid present with sodium tetraborate, producing a pink color in the presence of meta-hydroxydiphenyl which was quantified by spectrophotometry at 520 nm against a standard range of galacturonic acid from 10 to 100 mg/L (results were expressed in terms of galacturonic acid). Phenolic compounds were quantified by reaction in the presence of potassium ferricyanide, forming colored compounds detected at 715 nm against a standard range of gallic acid from 40 to 120 mg/L.

A CPT-1 inhibiting methanolic extract was obtained by extracting the leaves of the Averrhoa carambola plant using a methanol extraction scheme. Briefly, leaves of Averrhoa carambola were first manually ground into small particles resulting in a powder. The ground powder was then extracted with 25% methanol. Alternatively, the homogenized plant material can be combined with an equivalent volume of methanol, shaken in a sealed container for 30-120 minutes at 200 rpm, and centrifuged for 10 min, in order to form a clear methanol layer in the upper phase. After filtering and vacuum evaporation, the total concentrated extract was lyophilized (or concentrated by other means known in the art) to give an methanolic extract of Averrhoa carambola.

For sampling purposes, Averrhoa carambola leaf extracts were dispersed in 25% methanol at approximately 5 mg/mL. The soluble UV-absorbing components were separated and analyzed by HPLC. The HPLC instrument column was Zorbax SB-C18, 7.5 cm long×4.6 mm I.D. stainless steel, 3.5 micron particle size, and the detector was diode array UV absorbance, at 260 nm, 300 nm, and 360 nm. The HPLC analysis conditions were as follows:

Mobile Phase Gradient:

| | |
|---|---|
| 0 Minutes | 15% methanol (Solvent B)/85% water |
| | with 1% acetic acid (Solvent A). |
| 10 Minutes | 95% methanol/5% water with 1% acetic acid. |
| 15 Minutes | 95% methanol/5% water with 1% acetic acid. |
| 15.01 Minutes | 5% methanol/85% water with 1% acetic acid. |
| 19 Minutes | 15% methanol/85% water with 1% acetic acid. |

Flow Rate: 1.5 mL/min.

Column Temperature: 40° C.

Sample Injection Volume: 20 μL

Time of Run: 19 min

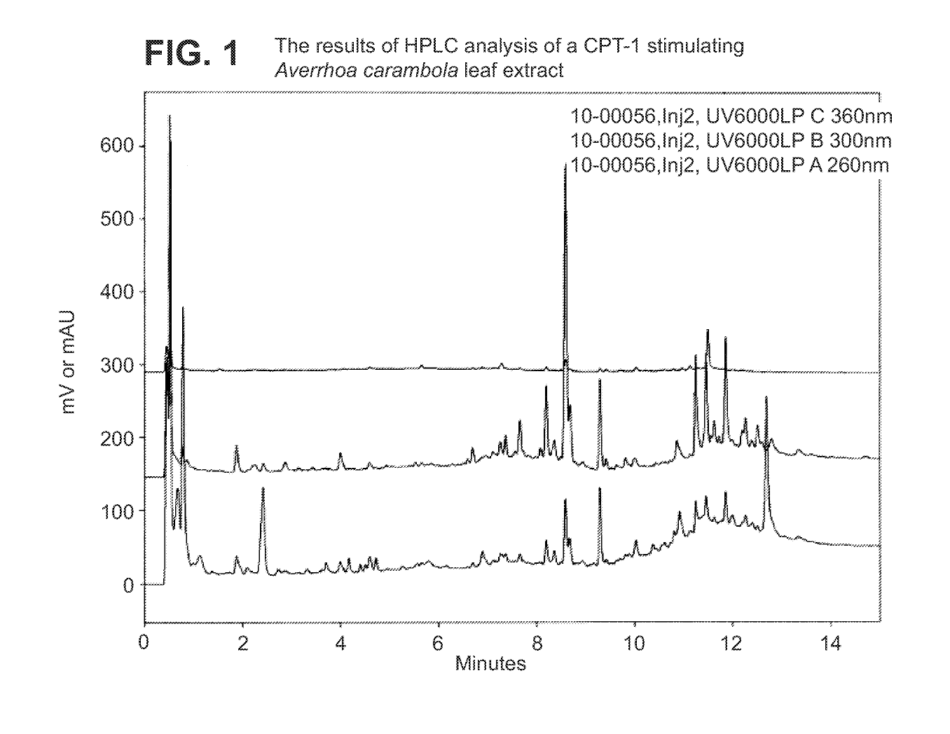

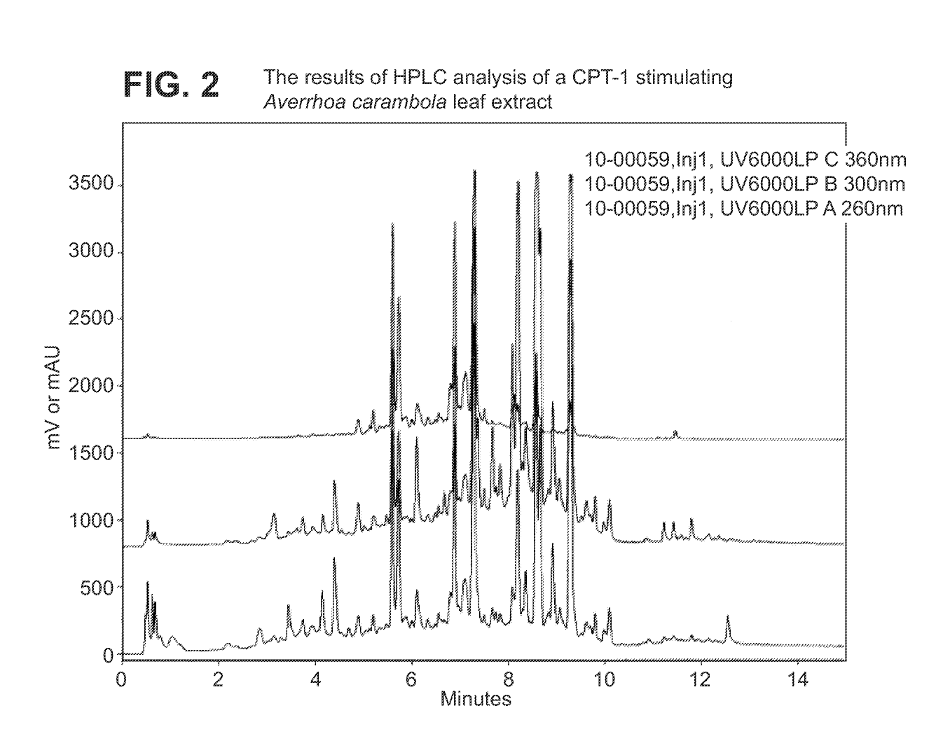

The HPLC spectra of a sample of an aqueous CPT-1 stimulating Averrhoa carambola leaf extract (FIG. 1) and a methanolic, CPT-1 inhibiting Averrhoa carambola leaf extract (FIG. 2) show different peak patterns under the same elution conditions, indicating chemical differences between the two extracts.

### Example 2

### Stimulation of CPT-1 Gene Expression by Averrhoa carambola Leaf Extract

Human pre-adipocytes were allowed to differentiate into adipocytes in Adipocyte Differentiation Medium for 7 days. On Day 8, Adipocyte Differentiation Medium was replaced with Adipocyte Maintenance Medium containing Averrhoa carambola leaf extract for another 7 days as described above. Averrhoa carambola leaf extract at the indicated weight percentages was added every other day. At the end of treatment, RNA was extracted from the adipocytes using RNA Easy mini kit (Qiagen, CA). 200 ng of total RNA was used to generate 20 microL of cDNA using High Capacity cDNA Reverse Transcript Kit (Applied Biosystem: Cat#4368814). Reverse transcriptase, Buffer, dNTP, Random primer, and RNase Inhibitor were diluted with the RNA according to the protocol from the manufacturer. One microliter of cDNA was used in 20 microL RTq-PCR reactions. Briefly, 10 μl of TaqMan Gene Expression Master Mix (Applied Biosystem; Cat#4369016), 8 μl of H₂O, 1 μl of cDNA, and 1 μl of either CPT-1 primer (Applied Biosystem; Hs03046298_s1) or 18S (Applied Biosystem; 4333760-1001032) as a house keeping gene were mixed in a 96 well polypropylene plate (Agilent Technologies; Cat#410088). RTq-PCR conditions were an incubation step at 50° C. for 2 minutes and an enzyme activation step at 95° C. for 10 minutes; followed by 45 cycles of 95° C. for 30 seconds and 60° C. for 1 minute. CT value was obtained from the software of the Stratagene MX2005P.

All samples were run in triplicate and normalized to 18S, and results were expressed as a percentage of the control, as presented in Tables 1 and 2.

TABLE 1
CPT-1 Gene Expression

| Concentration of Averrhoa carambola leaf extract | | |
|---|---|---|
| (stimulating)(aqueous extraction) | % change | p value |
| 0.6 % | 40.91% | 0.042 |
| 0.2 % | 30.10% | 0.037 |

TABLE 2
CPT-1 Gene Expression

| Concentration of Averrhoa carambola leaf extract | | |
|---|---|---|
| (inhibiting)(methanol extraction) | % change | p value |
| 0.4% | −10.38% | 0.249 |
| 0.2% | −34.33% | 0.003 |

Human adipocytes treated with the indicated weight % of aqueous Averrhoa carambola leaf extract showed a significant % increase in CPT-1 gene expression with the aqueous Averrhoa carambola leaf extract, as indicated in Table 1. In marked contrast, a methanol Averrhoa carambola leaf extract showed a decrease in CPT-1 gene expression, as indicated in Table 3. This finding is surprising and it indicates that the two Averrhoa carambola leaf extracts, obtained via aqueous extraction (data summarized in Table 1), or obtained via methanol extraction (data summarized in Table 2), have different chemical compositions.

### Example 3

### Reduction of Intracellular Triglycerides by Averrhoa carambola Leaf Extract

Cryopreserved human primary pre-adipocytes harvested from the subcutaneous adipose tissue of a healthy female were obtained from Zen-Bio (Research Triangle Park, N.C.). Following the manufacturer's instructions, the pre-adipocytes were cultured in Preadipocyte Medium containing DMEM/Ham's F-12 (1:1, v/v), HEPES (pH 7.4), fetal bovine serum, penicillin, streptomycin, and amphotericin B (Zen-Bio), in a humidified 37° C. incubator with 5% CO₂. After reaching 90% confluence, the pre-adipocytes were allowed to differentiate into adipocytes by adding Adipocyte Differentiation Medium containing DMEM/Ham's F-12 (1:1, v/v), HEPES pH 7.4, fetal bovine serum, biotin pantothenate, human insulin, dexamethasone, isobutylmethylxanthine, penicillin, streptomycin, and amphotericin B (Zen-Bio).

To treat adipocytes with Averrhoa carambola leaf extract, the aqueous extract (obtained according to the process of Example 1) was dissolved in Adipocytes Differentiation Medium (at two concentrations) and then added into cell culture for 7 days. The un-treated adipocytes were used as a control. After 7 days of incubation, Adipocytes Differentiation Medium was replaced with Maintenance Medium containing Averrhoa carambola leaf extract, DMEM/Ham's F-12 (1:1, v/v), HEPES pH 7.4, fetal bovine serum, biotin pantothenate, human insulin, dexamethasone, penicillin, streptomycin, and amphotericin B, and the adipocytes continued under incubation for another 7 days. The production of triglyceride, in the adipocytes was determined by using a triglyceride assay kit (Zen-Bio). Briefly, adipocytes were rinsed with a wash buffer and lysed in a lysis buffer following medium removal. Intracellular triglycerides were released into the lysis buffer and converted into glycerol-1-phosphate, which was subsequently oxidized to di-hydroxyacetone phosphate and hydrogen peroxide. Hydrogen peroxide was reacted with 4-aminoantipyrine (4-AAP) and sodium N-ethyl-N-(3-sulfopropyl)-m-anisidine (ESPA) to generate a quinoneimine dye, which shows an absorbance maximum at 540 nm. The increase in absorbance at 540 nm is directly proportional to the intracellular levels of triglycerides in the adipocytes. Results are presented below in Table 3.

TABLE 3
Intracellular Triglyceride Levels

| Concentration of Averrhoa carambola leaf extract | % Change | p value |
|---|---|---|
| 0.6% | −30.92% | 0.00 |
| 0.2% | −19.67% | 0.03 |

Human adipocytes treated with the indicated weight % of Averrhoa carambola leaf extract showed a significant % decrease in intracellular triglyceride levels, as indicated in Table 3.

### Example 4

### Exemplary Compositions

A sample oil-in-water emulsion cosmetic composition according to the invention was formulated according to Table 4.

TABLE 4
Sample Cosmetic Composition

| | | Preferred |
|---|---|---|
| | Concentration | Concentration |
| Ingredient | Range | Range |
| Aesthetic modifier | 0.1-2% | 0.5-2% |
| Emollient | 0.1-50% | 1-30% |
| Emulsifier | 0.5-30% | 0.5-10% |
| Anti-inflammation agent | 0.01-20% | 0.01-10% |
| Chelater | 0.01-20% | 0.01-5% |
| Coolant | 0-20% | 0.01-5% |
| Elastin stimulator | 0.01-2% | 0.1-1% |
| Exfoliator | 0.5-10% | 0.5-8% |
| Fragrance | 0-20% | 0.01-2% |
| Humectant | 0.01-30% | 0.01-25% |
| Microcirculation enhancer | 0.0001-2% | 0.01-1% |
| Neutralizer | 1-5% | 0.5-5% |
| Preservative | 0.01-20% | 0.01-5% |
| Sunscreen | 1-30% | 0.5-25% |
| Collagenase/elastinase inhibitor | 0.01-2% | 0.01-1% |
| Hawthorne (Crataeg. monog.) | 0-10% | 0.01-1% |
| Fruit. Extract | | |
| Coffee Seed Extract | 0-10% | 0.01-1% |
| Soybean (Glycine soja) Extract | 0-10% | 0.01-1% |
| Celosia cristata Extract & | 0-10% | 0.01-1% |
| Prunella vulgaris Extract | | |
| L-Carnitine Hydrochloride | 0-10% | 0.01-1% |
| Averrhoa carambola Leaf | 0.01-10% | 0.1-2% |
| Extract | | |
| Demineralized water | adjusted to 100% | adjusted to 100% |

The Averrhoa carambola Leaf Extract was an aqueous CPT-1 stimulating Averrhoa carambola leaf extract obtained as described in Example 3. Certain optional ingredients are indicated by a range including zero percent.

Other cosmetic compositions comprising an extract of Averrhoa carambola for topical application to the skin may be formulated by methods known in the cosmetic arts. A cream comprising an extract of Averrhoa carambola may optionally be administered along with a cosmetic composition of a gel for optimal results. Suitable ingredients for such formulations are found in the INCI Ingredient Dictionary and Handbook, 11th Edition, 2006, and in the International Cosmetic Ingredient Dictionary and Handbook, 10th Edition (2004), published by the Cosmetic, Toiletry, and Fragrance Association (CTFA), the disclosures of which are hereby incorporated by reference in their entirety.

### Example 5

### Consumer Study

A monadic, double blind, consumer home use test was conducted using a composition according to the invention (as disclosed in Table 4) over a four-week period among 120 women ages 25-59, in five geographically-dispersed locations in the United States. This study was executed by an independent consumer research agency, in accordance with consumer use test best practices and ASTM International guidelines for claims support. The respondents received instructions to use the composition at home twice a day for four weeks and were interviewed via telephone after 1 week, 2 weeks, and 4 weeks of twice-daily composition use. All data were returned to the independent research agency for processing and analysis using ANOVA. According to the results of the study, 61% of the respondents agreed that the composition dramatically reduce of the look of cellulite; 72% of the respondents agreed the composition made their skin feel more toned and they had the freedom and confidence to wear what they wanted; 66% of the respondents agreed that the depth and intensity of their cellulite was reduced in just two weeks; and 59% of the respondents agreed that even their most stubborn cellulite was visibly improved in just two weeks. These results were significant at a 90% confidence interval.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A method for treating a skin condition characterized by excess lipids, comprising topically applying to skin in need thereof an effective amount of a Carnitine Palmitoyl Transferase-1 (CPT-1) stimulating aqueous extract of the leaf of Averrhoa carambola in a cosmetically acceptable vehicle for a time sufficient to improve the appearance of said skin.

2. The method according to claim 1, wherein said aqueous extract has an HPLC analysis according to FIG. 1

3. The method according to claim 1, wherein said aqueous extract comprises less than 0.1% by weight phenolic compounds and at least 20% by weight carbohydrates.

4. The method according to claim 1, wherein the skin condition is cellulite.

5. The method according to claim 1, wherein the skin condition is acne.

6. The method according to claim 1, wherein the skin condition is oily skin.

7. The method according to claim 1, wherein said aqueous extract further comprises at least one other anti-lipid agent.

8. The method according to claim 7, wherein said at least one other anti-lipid agent comprises at least one agent selected from the group consisting of a phosphodiesterase inhibitor, an adenylate cyclase activator, a lipolysis stimulator, a beta-adrenergic receptor agonist, an alpha-2-adrenergic receptor antagonist, and combinations thereof.

9. The method according to claim 7, wherein said at least one other anti-lipid agent comprises at least one agent selected from the group consisting of a xanthine analog, forskolin, a forskohlii extract, a hawthorne extract, a cola extract, isoproterenol, yohimbine, Ginkgo biloba extract, perilla oil, and combinations thereof.

10. (canceled)

11. The method according to claim 1, wherein said time sufficient to improve the appearance of said skin comprises a period of at least 2 weeks, and wherein said aqueous extract is applied at least once a day.

12. (canceled)

13. (canceled)

14. A composition for treating a skin condition characterized by excess lipids, comprising an effective amount of a CPT-1 stimulating aqueous extract of the leaf of Averrhoa carambola in a cosmetically acceptable vehicle.

15. The composition according to claim 14, wherein said composition further comprises at least one other anti-lipid agent selected from the group consisting of a xanthine analog, forskolin, a forskohlii extract, a hawthorne extract, a cola extract, isoproterenol, yohimbine, Ginkgo biloba extract, perilla oil, and combinations thereof.

16. (canceled)

17. (canceled)

18. (canceled)

19. A composition for treating a skin condition characterized by excess lipids, comprising an effective amount of a CPT-1 stimulating aqueous extract of the leaf of Averrhoa carambola in a cosmetically acceptable vehicle, wherein said aqueous extract comprises less than 0.1% by weight phenolic compounds and at least 20% by weight carbohydrates.

20. The use of an aqueous extract of the leaf of Averrhoa carambola for the treatment of excess lipids, wherein said aqueous extract comprises less than 0.1% by weight phenolic compounds and at least 20% by weight carbohydrate.

21. The use of claim 20, wherein the treatment is comprised of the upregulation of CPT-1 expression.

22. The use of claim 21, wherein the upregulation of CPT-1 expression is assayed by a measurable increase in CPT-1—associated mRNA transcription.

23. The use of claim 22, wherein the upregulation of CPT-1 expression is assayed by a measurable increase in CPT-1—associated protein expression.

24. (canceled)

25. (canceled)

26. (canceled)

27. (canceled)

28. (canceled)

29. (canceled)

30. (canceled)

31. (canceled)

32. The use of claim 20, wherein the aqueous extract is obtained by aqueous solubilization of Averrhoa carambola leaf powder to form a mixture, followed by enzymatic hydrolysis of the mixture and separation of the aqueous phase of the mixture, resulting in said aqueous leaf extract of Averrhoa carambola.

33. The use of claim 20, wherein said aqueous extract has an HPLC analysis according to FIG. 1.
